(19) United States
(12) Patent Application Publication
(10) Pub. No.: US 2008/0045883 A1
(43) Pub. Date: Feb. 21, 2008

(54) Devices and methods for monitoring and delivering therapeutics to the spinal cord

(76) Inventor: Milan Radojicic, Santa Rosa, CA (US)

Correspondence Address: Milan Radojicic, 4145 Shadow Lane, Apt. 435, Santa Rosa, CA, 95405, US

(21) Appl. No.: 11/840,213

(22) Filed: Aug. 16, 2007

Related U.S. Application Data
(60) Provisional application No. 60/822,640, filed on Aug. 17, 2006.

(51) Int. Cl. A61M 1/00; A61B 5/00; A61M 31/00

(52) U.S. Cl. 604/28; 600/561; 604/508

(57) ABSTRACT

This invention is a continuous monitor of the spinal cord and brain microenvironment in injury and disease that also allows therapeutic interventions. It utilizes a multiport catheter that contains a transducer at the tip for monitoring spinal physiological parameters and also allows via additional ports for sampling and exchange of spinal fluid, as well as drug delivery to the central nervous system. This invention allows for more precise therapeutic interventions in spinal cord and brain injury and disease.

### CROSS-REFERENCE TO RELATED APPLICATIONS

This application claims the benefit of provisional patent application Ser. No. 60/822,640, filed 2006 Aug. 17 by the present inventor.

### FEDERALLY SPONSORED RESEARCH

Not applicable.

### SEQUENCE LISTING OR PROGRAM

Not applicable.

### BACKGROUND OF THE INVENTION

1. Field of the Invention

The present invention relates generally to surgical methods and medical devices. More particularly, it concerns methods and devices for monitoring and delivering therapeutics to the spinal cord and brain along a spinal fluid pathway or cistern, particularly the lumbar cistern.

2. Prior Art

By the end of the next decade, 300,000 people will be living with chronic spinal cord injury in the US alone. Advances in medical and rehabilitative care have improved survival rates for these individuals, but many experience clinical decline even years after the initial injury. Clinical decline is often accompanied by a slow and progressive cavitation of the central spinal cord, known as post-traumatic syringomyelia. The pathogenesis of this disease remains poorly understood, but may be related to spinal cord edema and altered intraspinal pressure due changes in spinal fluid homeostasis.

Beyond spinal cord injury, other spinal cord diseases may similarly be impacted by such pathophysiological processes. Such diseases include spinal tumors and vascular malformations, spinal infections, multiple sclerosis, transverse myelitis, and non-traumatic syringomyelia. Moreover, considerable morbidity and mortality may results from spinal surgical interventions where monitoring has been heretofore limited to the electrophysiological parameters.

Following spinal cord injury and disease, normal CSF dynamics may be distorted by a number of possible mechanisms, including subarachnoid CSF outflow obstructions, changes in compliance of the subarachnoid space, or elevated intraspinal pressures. Altered CSF dynamics are believed to result in localized spinal cord edema, known as the presyrinx state that subsequently gives rise to central canal dilation and/or the formation of intraspinal glial-lined parenchymal cysts.

The propagation of intraspinal cavities requires a driving force sufficient to propel fluid via a one-way valve mechanism into the cysts, which often contain fluid at a higher pressure than the subarachnoid space. Proposed driving forces include cardiac pulsations along vessels, postural changes and valsalva movements and elevated intraspinal pressures. I have additionally suggested consideration of the transient hypertensive episodes of autonomic dysreflexia as a potential driving force.

The measurement and treatment of raised intracranial pressures following insults to the brain is very well established and is based on many years of work in experimental neurology establishing a pressure-volume relationship in cranial compartment, first suggested by Alexander Monro in 1783. With the increasing recognition on the role of spinal edema, pressure and altered cerebrospinal fluid dynamics on spinal cord injury and disease, it would stand to reason that modern should have a means of monitoring such phenomena. Indeed, continuous monitoring of various physiological parameters is a mainstay of modern critical care. Prior art has focused on intracranial pressure monitoring, for example see Beckman et al. U.S. Pat. No. 5,325,865. Purdy et al. U.S. Pat. No. 7,150,737 B2 would allow for navigating the subarachnoid space, but limit interventions to heating and cooling of the nervous tissue. However, until this Applicant's invention, no such devices and methods for continuous monitoring of several spinal physiological parameters, along with methods of drug delivery and therapeutics, have existed.

### DETAILED DESCRIPTION OF THE INVENTION

This invention is a continuous monitor of the spinal cord and brain microenvironment in injury and disease that also allows therapeutic interventions. It utilizes a multiport catheter that contains a transducer at the tip for monitoring spinal physiological parameters and also allows via additional ports for sampling and exchange of spinal fluid, as well as drug delivery to the central nervous system. This invention allows for more precise therapeutic interventions in spinal cord and brain injury and disease.

While the above description contains many specificities, these should not be construed as limitations on the scope of the invention, but as exemplifications of the presently preferred embodiments thereof. Many other ramifications and variations are possible within the teaching of the invention. Additionally, any combination of the above examples may be possible.

Thus the scope of the invention should be determined by the appended claims and their legal equivalents, rather than the examples given.

1. A method of treating the central nervous system in injury and disease in a vertebrate organism by providing a catheter having a proximal end, a distal end, and an intervening elongate shaft that is placed along a spinal fluid pathway or cistern, particularly the lumber cistern

2. A method of claim 1 wherein said catheter is placed extadurally

3. A method of claim 1 wherein said catheter is placed intradurally

4. A method of claim 1 where said catheter has at least one port

5. A method of claim 4 where one port contains a transducer at the distal end of the catheter

6. A method of claim 4 where one port contains a biosensor at the distal end oil the catheter

7. A method claim 4 where one port is utilized for outflow of fluid

8. A method of claim 4 wherein one port is utilized for inflow of fluid

9. A method of claim 4 wherein at least one port is used for introduction of a surgical tool

10. A method of claim 7 wherein the outflow tract is used for delivery of a therapeutic substance to the cerebrospinal fluid for treatment of a brain or spinal cord disease

11. A method of claim 5 wherein said transducer can transmit and collect electromagnetic radiation, particularly in the ultrasonic and infrared range, but also visible range

12. A method of claim 11 wherein said transducer continuously monitors structural characteristics of the spinal cord, such as central canal size and parenchymal edema

13. A method of claim 11 wherein said transducer continuously monitors flow characteristics of the spinal cord, including blood and cerebrospinal fluid velocity

14. A method of claims 2 and 5 wherein said transducer is an applanation pressure sensor

15. A method of claim 3 and 5 wherein said transducer can monitor spinal fluid pressure

16. A method of claims 3 and 5 wherein said transducer can monitor spinal dural compliance

17. A method of claims 12 and 13 wherein the physiologic parameters of a patient are monitored pre- and post-valsalva maneuver in order to compare to responses in control subjects

18. A method of claims 7 and 8 wherein cerebrospinal fluid is drained and exchanged with an inflow of artificial cerebrospinal fluid

19. A method of claim 7 and 8 wherein cerebrospinal fluid is drained, treated (e.g., dialyzed of a substance) and re-circulated into the subarachnoid space

20. A method of claim 3 and 7 wherein cerebrospinal fluid is monitored for physiological parameters, such as pH, glucose, protein, cell count and CSF gas values
